(19) United States
(12) Patent Application Publication
(10) Pub. No.: US 2013/0041104 A1
(43) Pub. Date: Feb. 14, 2013

(54) CONJUGATES OF 18F CARRIERS HAVING BIOACTIVE, ORGANIC COMPOUNDS AND THE PREPARATION THEREOF

(71) Applicant: Matteo Scabini, Seregno (IT)

(72) Inventor: Matteo Scabini, Seregno (IT)

(73) Assignee: APTENIA S.R.L., Milano (IT)

(21) Appl. No.: 13/521,461

(22) PCT Filed: Jan. 17, 2011
(86) PCT No.: PCT/DE2011/000042

§ 371 Date: Jul. 11, 2012

(30) Foreign Application Priority Data
Feb. 6, 2010 (DE) 10 2010 007 097.1

(51) Int. Cl. C07F 17/00; C08G 83/00; C07K 1/00; C07H 19/073; C07H 21/00

(52) U.S. Cl. 525/50; 556/41; 536/28.54; 536/23.1; 530/408

(57) ABSTRACT

The invention relates to novel chemical compounds, which can be fluorinated with ¹⁸F under especially mild conditions. The novel chemical compounds thereby enable the use of a novel fluorination method according to the invention, wherein the substrate to be fluorinated is immobilized on a polymer during the fluorination. The method is characterized in that the method requires fewer and simpler manipulations than methods of the prior art. The occupational safety in the laboratory or hospital is thereby increased especially during work with the radionuclide ¹⁸F.

Among the methods used in radiology, positron emission tomography (PET) is a preferred method of tracking disease courses and the therapy thereof in many indication areas, e.g. in oncology. The fluorine isotope ¹⁸F, prepared synthetically in a cyclotron, is preferred for this. Labeled compounds, which are absorbed selectively by the pathologically altered tissue or which can bind selectively to pathologically altered cells, are prepared with it. Such ¹⁸F-labeled compounds are denoted hereinafter as PET “tracers”. They permit, by means of autoradiography, visualization of the tissue to which they bind or in which they are absorbed. By positron emission tomography (PET) with PET “tracers”, the diseased tissue in the body of the patient can be localized and its size determined.

Among the positron-emitting radionuclides, the fluorine isotope ¹⁸F is preferred, because it can be introduced into many organic compounds, which can then be used as PET “tracers”. Advantageous for this is the relatively long half life of 109.6 minutes and a low-energy β+ emission (635 keV) of this nuclide.

The best-known example of such a preparation is 2-¹⁸F-fluorodeoxyglucose (¹⁸FDG) (J. Nucl. Med. 1978, 19, 1154-1161).

Recently, short synthetic nucleic acids or peptides, known as aptamers, which can bind selectively to the surfaces of pathologically altered cells, have also been used when labeled with ¹⁸F. (Friebe, Matthias et al. PCT Int. Appl. WO2009033876 A1). For example, aptamers prepared such that they are able to bind tumor markers have recently been used in oncology. (Hoppe-Seyler F, Butz K., J. Mol. Med. 2000, 78, 426-30; The Aptamer Handbook: Functional Oligonucleotides and Their Applications, Sven Klussmann (Editor) ISBN: 978-3-527-31059-3 March 2006, Wiley-VCH Verlag GmbH & Co. KGaA). More selective PET can be performed with such novel aptamers.

The labeling of organic molecules with radioactive ¹⁸F often necessitates several wet-chemical steps, including some that must be carried out above 100° C., as well as laborious chromatography steps. Because of the high radioactivity, therefore, the commercial introduction of ¹⁸F is associated with high risks for the performing persons.

A further disadvantage of the use of the now firmly established ¹⁸FDG is artifacts caused by lack of selectivity of the diagnostic. Thus, for example, ¹⁸FDG is absorbed not only by tumor tissue but also by active skeletal muscles. (Seminar in Nuclear Medicine 2004, XXXIV, 2, 122-133).

The present invention relates to novel chemical compounds, which can be fluorinated under particularly mild conditions. It is the objective to obtain stable chemical bonds with fluorine atoms. They should be so stable under physiological conditions that the fluorinated compounds are suitable as “PET tracers”.

A further objective is to guarantee a high degree of work safety with a novel fluorination method according to the invention, involving minimum time and effort. This is achieved by the fact that the manipulations with radionuclides are minimized and the radioactive waste is produced in concentrated form.

A further objective of the invention is to conjugate, with ¹⁸F, using a generally applicable, safe, rapid method, bioactive molecules that bind special tissue types, such as tumors.

Thus it is simpler than heretofore to use molecules as selective ¹⁸F “PET” tracers.

The novel fluorination method and conjugation method according to the invention are carried out on a special polymer. In the process, the substrate to be fluorinated is first immobilized on a polymer. Thereafter the fluorination of the immobilized substrate takes place on solid phase. Surplus radionuclides are eluted from the polymer. Finally the conjugation reaction takes place with a bioactive compound, which is able to bind special tissue types, such as tumors. During the conjugation reaction, a Staudinger reaction, the finished ¹⁸F-labeled “PET tracer” is eliminated from the solid phase.

The Carrier:

The conjugation reaction or labeling reaction takes place on a carrier “Pol” (Scheme 1) that is insoluble in aqueous and organic solutions. The preferred carrier materials are constituted such that they are able to react at their conjugated functional groups and/or molecules with other molecules, regardless of whether the carrier is immersed in organic or in aqueous solutions thereof. (Scheme 1)

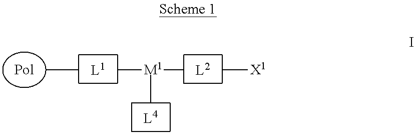

Scheme 1: The insoluble carrier “Pol” with the groups L¹, L² and L⁴; M¹ is a heteroatom (preferably P) on which a Staudinger coupling can occur; X¹ is an acylatable group, preferably SH, NH or OH.

Pol:

Herein “Pol” (Scheme 1) is an insoluble organic or inorganic polymer, a metal surface or macromolecular surface on which chemical reactions can be carried out in organic and aqueous media.

Among the organic polymers, the following materials, for example, can be used as homopolymers or copolymers in all possible variations: polystyrene, polyethylene, polypropylene, polyphenylene, polyacrylamides, polyacrylic esters, polyamides, polysulfones, oligosaccharides, e.g. dextran, PEG, polypropylene glycol, as well as all partially or exhaustively fluorinated derivatives of the aforesaid materials.

Preferred are polymers similar or identical to the “Pol” Tentagel® (Rapp Polymere GmbH, Ernst-Simon-Str. 9, D72072 Tübingen, Germany), polymer bodies produced by “injection molding”, e.g. (Mimotopes Pty Ltd, 11 Duerdin Street, Clayton Victoria 3168, Australia, www.mimotopes.com) or materials that consist of ferromagnetic substances.

L¹:

The “L¹” group is bound covalently or by strong ionic or Van der Waals interactions to the aforesaid carrier materials.

L¹ consists of 1-200 carbon atoms and/or 0-200 heteroatoms, which can occur in aromatic rings, heteroaromatic rings, aliphatic rings, heteroaliphatic rings, aliphatic chains. For L¹ it is possible to use heteroatoms from the following list in all possible combinations: F, Cl, Br, I, S, O, N, Se, Te, Si, Al, Ge, P, As, Sb, B, Li, Na, K, Cs, Ca, Mg, Sr, Ba, Fe, Ce, Sn, H and the isotopes thereof.

L²:

L² consists of 1-200 carbon atoms and/or 0-200 heteroatoms, which can occur in aromatic rings, heteroaromatic rings, aliphatic rings, heteroaliphatic rings, aliphatic chains and heteroaliphatic chains.

For L² it is possible to use heteroatoms from the following list in all possible combinations: F, Cl, Br, I, S, O, N, Se, Te, Si, Al, Ge, P, As, Sb, B, Li, Na, K, Cs, Ca, Mg, Sr, Ba, Fe, Ce, Sn, H and the isotopes thereof.

However, L² groups of the general formulas —CR¹²R¹³— or (—CR¹²R¹³—CH₂—) are preferred, wherein the moieties R¹² and R¹³ may be H or short alkyl groups with up to 20 C atoms but also phenyl, and partly or exhaustively fluorinated derivatives thereof.

In special embodiments the C atoms of the moieties R¹² and R¹³ are bridged with one another and part of a cyclopropyl, cyclobutyl, cyclopentyl, cyclohexyl ring as well as heterocyclic derivatives thereof that contain O and S atoms. See Formulas XI and XII. (Scheme 2).

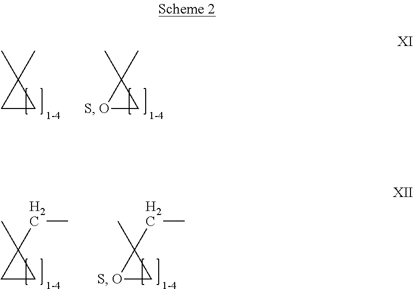

Scheme 2: Some preferred L² groups

X¹:

X¹ represents an acylatable group. This also includes compounds in which L²-X¹ can be bound in a heterocycle with 1-10 carbon atoms and/or 0-10 heteroatoms. The heteroatoms used here are N, S, O. In preferred embodiments, X¹ is SH, OH, NH, the silylated or stannylated derivatives thereof.

M¹:

M¹ is a trivalent atom that carries a free electron pair. This includes P, As, Sb and Bi.

L⁴ is a moiety that consists of 1-200 carbon atoms and/or 0-200 heteroatoms, which may occur in aromatic rings, heteroaromatic rings, aliphatic rings, heteroaliphatic rings, aliphatic chains and heteroaliphatic chains.

L⁴may contain heteroatoms from the following list: F, Cl, Br, I, S, O, N, Se, Te, Si, Al, Ge, P, As, Sb, B, Li, Na, K, Cs, Ca, Mg, Sr, Ba, Fe, Ce, Sn, H and the isotopes thereof.

The General Polymeric Conjugation Reagent III.

In the first step the loading of the carrier “I” takes place with a labeled compound “II” or with a compound “IV”, which according to the invention may be fluorinated.

The moiety X¹ of the material described in Formula I may be esterified with an ¹⁸F-labeled carboxylic acid or a carboxylic acid molecule that can be labeled with ¹⁸F. Examples for ¹⁸F-labeled known carboxylic acids or acylatable derivatives “II” thereof (Scheme 3) are listed in the following publications (Bayer Schering Pharma Aktiengesellschaft, Germany. PCT Int. Appl. 2008, 236 pp. Application: WO 2007-EP8042 20070907. Priority: EP 2006-90166 20060908; EP 2007-90079 20070423.9 or Mu et al. Angew. Chem. Int. Ed. 2008, 47, 4922-4925)

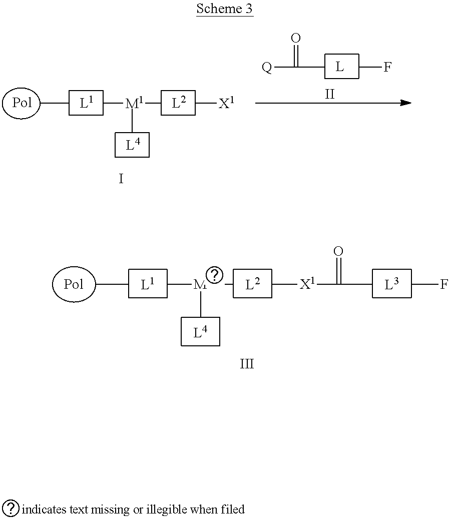

Scheme 3: The acylation of carrier “I” with an already existing ¹⁸F-labeled acid “II”.

In this way a general polymeric conjugation reagent may be obtained. A special advantage of the described method lies in the fact that the general polymeric conjugation reagent “III” may be packed into a chromatography column. All chemical transformations depicted in the following may therefore be carried out in this chromatography column under surplus of the reagent to be added. In this way high yields may be achieved and surplus reagents and secondary products may be eluted by washings with appropriate solvents.

Alternatively to this, the polymer body “III” may be produced by “injection molding” and immersed in the appropriate reagents for subsequent transformations. Surplus reagents may be washed away easily by immersion in rinse solutions.

The execution of the method according to the invention in order to obtain general polymeric conjugation reagent “III” takes place by means of acylation of the carrier “I” with a carboxylic acid according to the invention or the acylatable derivative “IV” thereof. It carries at least one functional “X²” group into which ¹⁸F may be introduced. From the carrier “V” preparable in this way, the conjugation reagent “III” may be easily obtained in the following. (Scheme 4).

Compounds of the general formula “IV” may be prepared by analogy with the following literature procedures. (Andreas Gansaeuer et al. J. Am. Chem. Soc. 2005, 127, 11622-11623; Li Ming Gao et al. J. Biol. Inorg. Chem. 2007, 12, 959-967)

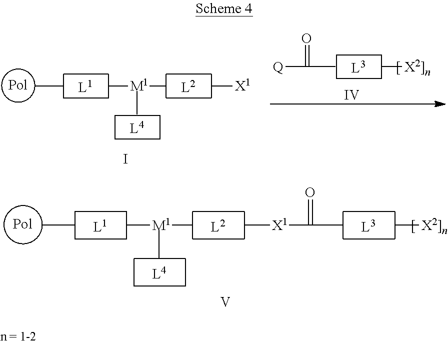

Scheme 4: The acylation of carrier “I” with an unlabeled acid “IV”, which carries a group into which ¹⁸F may be selectively introduced.

Scheme 5 shows a fluorine-affine group that may be used according to the invention.

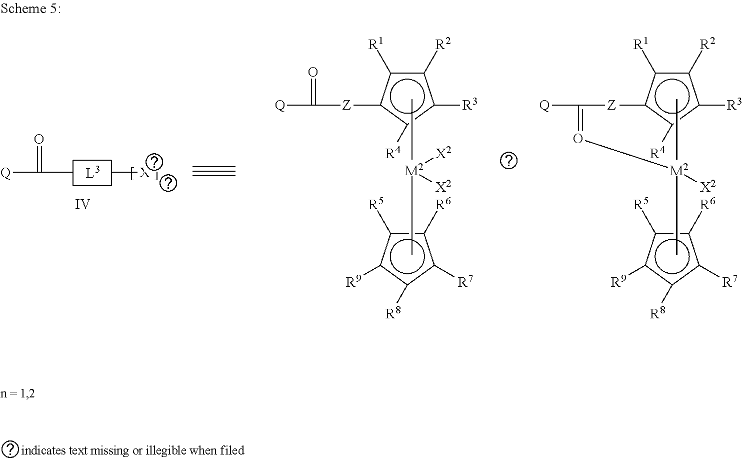

Scheme 5: The fluorine-affine group based on an element of the 4th group of the periodic system (M²=Ti, Zr, Hf)

Z:

Herein “Z” is a group of the type R¹-R⁹, which carries a (C═O)Q group and represents the moiety of L3 without the fluorophilic group.

R¹-R⁹:

The R¹-R⁹ moieties, which while variable independently of one another are H atoms or organic moieties, which consist of 1-200 carbon atoms and/or 0-200 heteroatoms, which can occur in aromatic rings, heteroaromatic rings, aliphatic rings, heteroaliphatic rings, aliphatic chains and heteroaliphatic chains. Moreover, neighboring moieties may be bridged by bridges of carbon and heteroatoms.

The following may be used as heteroatoms: F, Cl, Br, I, S, O, N, Se, Te, Si, Al, Ge, P, As, Sb, B, Li, Na, K, Cs, Ca, Mg, Sr, Ba, Fe, Ce.

X²

X² is a group that may be substituted by fluoride. This includes, for example, the following heterocycles in unsubstituted or substituted form: imidazole, triazoles, tetrazole, thiazole, benzimidazole, benzotriazole, indole, imidazoles, aniline, pyrazole, pyrrolidone, azetane, azepine, benzodiazepine, piperidine, purine, morpholine, piperazine, triazine, oxazole, hydantoin, aziridine, pyrrolidine, pyrrole, hexamethyleneimines, azaindole or a group, the conjugated organic or inorganic acids of which have a pKa<12. Among those phenoxy, alkoxy, thiophenoxy or thioalkoxy groups substituted appropriately with halogens, pseudohalogens and nitro groups. Further, OH, SH, Cl, Br and I.

Q:

Q is a leaving group for acylations or a group made capable of acylation by reaction with coupling reagents that are usable for esterifications. (Methods of Organic Chemistry, E. Müller ed., Volume XV/1-2, “Synthesis of Peptides”, Georg Thieme Verlag 1974). This includes the following heterocycles in unsubstituted or substituted form: imidazole, triazoles, tetrazole, thiazole, benzimidazole, benzotriazole, indole, imidazoles, aniline, pyrazole, pyrrolidone, azetane, azepine, benzodiazepine, piperidine, purine, morpholine, piperazine, triazine, oxazole, hydantoin, aziridine, pyrrolidine, pyrrole, hexamethyleneimines, azaindole, hydroxysuccinimide, oxybenzotriazole or a group, the conjugated organic or inorganic acids of which have a pKa<12. Among those phenoxy, alkoxy, thiophenoxy or thioalkoxy groups substituted appropriately with halogens, pseudohalogens and nitro groups. Further, the OH, SH, Cl, Br, F and I groups.

The Fluorination on Solid Phase:

It is particularly advantageous when the column material “V” is brought to reaction with highly radioactive compounds, e.g. ¹⁸F-labeled compounds, or with ¹⁸F, wherein the radioisotope is immobilized on the column material.

Here the advantage of the moiety “L³” in compounds “IV” and “V” becomes apparent, in that it permits the introduction of the ¹⁸F by substitution of the group(s) “X²” under conditions that the carrier material tolerates.

Preferred in this case is an organodiyl “L³” to which an element of the 4th or 14th group of the periodic system is chemically bound. On each element of the 4th or 14th group there is or are located the one or two leaving group(s) “X²”, which is (are) substitutable by fluoride under mild conditions.

The case of the element of the 4th group of the periodic system with M²=Ti, Zr, Hf is illustrated in Scheme 5. The substitution of a preferred leaving group X² in Scheme 5 by fluoride is described in the following. (Laurianne Bareille et al. Eur. J. Inorg. Chem. 2005, 2451-2456)

The fluorination is carried out with fluoride ions, because all ¹⁸F-labeled compounds are preferably derived from the K¹⁸F prepared in the cyclotron.

During the fluorination, the column jacketing protects the operating personnel. Surplus radioactive material may be washed away from the polymer, and it arrives in very concentrated and easily separable form at the column outlet. In this way the conjugation reagent “III” can be obtained safely. (Scheme 6)

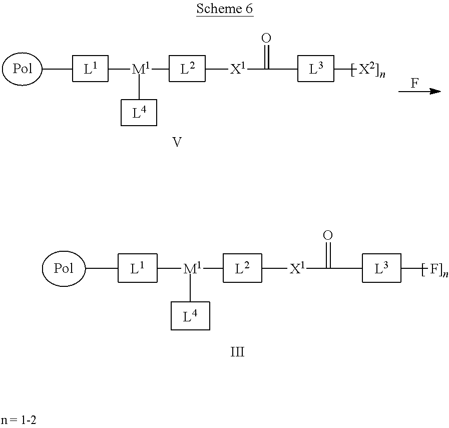

Scheme 6: The fluorination of carrier “V”. (Substitution of X² by ¹⁸F)

Also, the carrier “III” according to the invention either may be packed as a chromatography column or transported to the site as macroscopic polymer bodies prepared by “injection molding” without danger for the personnel, which increases its flexibility of application for the clinic. The fluorinated carrier, the fluorine-affine group of which is an element of the 4th group of the periodic system, is illustrated in scheme 7.

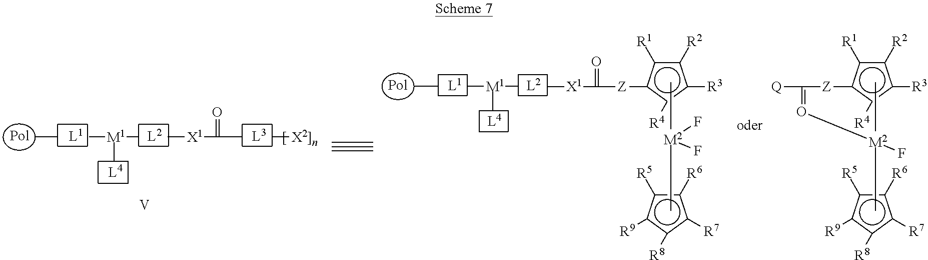

Scheme 7: The fluorine-affine group of the compound “V”. In this way fluorine may be added onto an element of the 4th group of the periodic system under formation of a very strong bond.

The Conjugation of a Bioactive Molecule:

Essential for the method according to the invention is the atom M¹ of the carrier “III” (Scheme 3, 4), which permits a Staudinger coupling with a bioactive organyl azide “VI”, which is able to bind selectively to specific cells, e.g. tumor cells (Scheme 8).

For this the general reagent “III”, which may be prepared according to Scheme 3 or according to the invention according to Scheme 6, is brought to reaction with a bioactive substance “VI”, which carries an azide group. (Scheme 8). On the basis of the Staudinger reaction occurring on the solid phase, the compound selectively recognizing special cell types, e.g. the tumor-binding ¹⁸F-labeled compound “IX”, may be eluted from the carrier and used as a diagnostic and/or therapeutic.

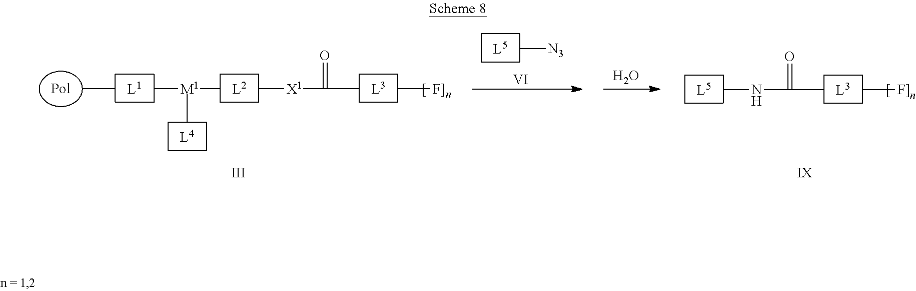

Scheme 8: The Staudinger reaction of the carrier “III” with an organyl azide “VI”.

Particularly advantageous in this case is application of the polymeric conjugation reagent “III” to an extremely broad multitude of tumor-binding bioactives “L⁵” “VI”. (Scheme 8)

Herein L⁵ is a bioactive substance, which may consist of an oligonucleotide of 0-200 natural nucleotides or synthetic analogs and isosteres, which as internucleotide bonds may contain: phosphorothioates, phosphorodithioates, phosphorotrithioates, phosphorotetrathioates, arsenates, thioarsenates, dithioarsenates, trithioarsenates, tetrathioarsenates, fluorophosphates, phosphorofluorothioates, phosphorofluorodithioates, phosphorofluorotrithioates, H-phosphonates, alkylphosphonates, arylphosphonates, H-phosphonothioates, alkylphosphonothioates, arylphosphonodithioates, H-phosphonodithioates, alkylphosphonodithioates, arylphosphonodithioates, H-phosphonotrithioates, alkylphosphonotrithioates, arylphosphonotrithioates, phosphoramides, phosphorodiamides, phosphorotriamides, phosphoroamidothioates, phosphoroamidodithioates, phosphorodiamidothioates, phosphorotriamidothioates, actals, ketals, phosphine oxides, phosphine sulfides, silyl groups, carboxylic acid amides, triazoles, oxadiazoles, sulfates, sulfamides, sulfonamides, disulfides, amine oxides, nitrones, as well as BH₃ adducts of the phosphites, thiophosphites, dithiophosphites, trithiophosphites, phosphoamidites, phosphodiamidites, triaminophosphines, phosphorothioamidites; alkylborane adducts of the phosphites, thiophosphites, dithiophosphites, trithiophosphites, phosphoamidites, phosphodiamidites, triaminophosphines, phosphorothioamidites, or dialkylborane adducts of the phosphites, thiophosphites, dithiophosphites, trithiophosphites, phosphoamidites, phosphodiamidites, triaminophosphines, phosphorothioamidites, further trialkylborane adducts of the phosphites, thiophosphites, dithiophosphites, trithiophosphites, phosphoamidites, phosphodiamidites, triaminophosphines, phosphorothioamidites in every possible combination.

However, the bioactive substance L⁵ may also be a synthetic polymer, which consists of 0 to 50 units of the ethylene, propylene, ethylene glycol, propylene glycol, acrylic ester, acrylamide, pyruvate, caprolactam, styrene groups, polyamines of various chain length, a polymer prepared by ROM and the arylated and alkylated derivatives thereof in all possible mixtures and combinations. This polymer may be substituted by aromatic rings, heteroaromatic rings, aliphatic rings, heteroaliphatic rings, aliphatic chains and heteroaliphatic chains and contain the heteroatoms F, Cl, Br, I, S, O, N, Se, Te, Si, Al, Ge, P, As, Sb, B, Li, Na, K, Cs, Ca, Mg, Sr, Ba, Fe, Ce, Sn, H and the isotopes thereof. Examples for such a synthetic polymer are described by Kleiner et al. J. Am. Chem. Soc. 2008, 130, 4646-4659, Heemstra et al., J. Am. Chem. Soc. 2009, 131, 11347-11349, Ura et al. Science (Washington) 2009, 325, 73-77.

However, the bioactive substance L⁵ may also consist of a peptide or protein of 0-2000 amino acids or the analogs, stereoisomers or isosteres thereof and contain e.g. D-amino acids, α,ω-amino acids taurine derivatives and PNA units.

Further, the bioactive substance L⁵ may be a carbohydrate of 0-50 saccharide units, the analogs or stereoisomers thereof.

Finally, L⁵ may be a bioactive low molecular weight molecule, a natural or synthetic protein agonist, antagonist, enzyme inhibitor or nucleic acid binder, a lipid of the group of the steroids, terpenes, macrolides, polyketides and/or the natural and synthetic derivatives thereof.

Scheme 9 shows a summary of the new labeling method according to the invention.

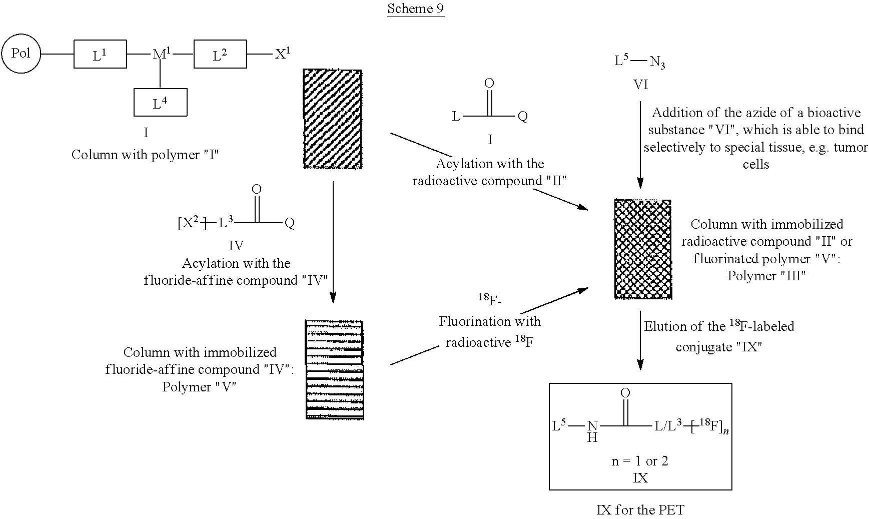

Scheme 9 shows the use of the labeling reagent “I” described according to the invention, packed in a column, which reagent may be esterified with carboxylic acids “II” and “IV”. In the case “II” the carboxylic acid is ¹⁸F-labeled, in the case “IV” it carries a fluorine-affine group. The latter may be fluorinated on the solid phase. The acylated carrier “III” may then be conjugated with a tumor-binding substance, e.g. an aptamer, wherein the conjugate “IX” is liberated.

### EXAMPLES

### Example 1

### Preparation of a Compound of the Type “V” (XVIII) with a Fluorine-Affine Group

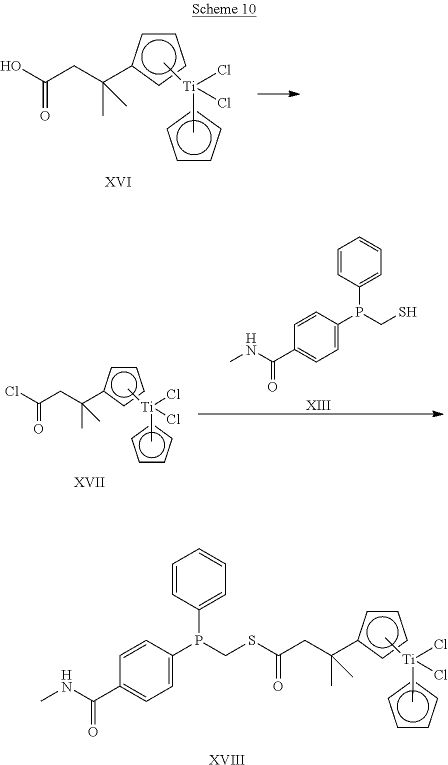

The titanocene carboxylic acid (Gansaeuer et al. J. Am. Chem. Soc. 2005, 127, 11622-11623.) is transformed to the acid chloride:

(313 mg, 1.0 mmol) of I is reacted with SOCl₂ (1.0 mL) within one hour at room temperature. Surplus SOCl₂ is distilled off. The residue is then dissolved in CH₂Cl₂ (5 mL) and transferred into a suspension of mercaptomethylphosphinyl tentagel (0.25 SH equivalents), (Hanyoung, Kim at al. Chem. Lett. 2006, 8, 1149-51) and DIPEA (120 mg, 5.0 mmol) in CH₂Cl₂ (5 mL). The reaction mixture is shaken for 16 hours at room temperature. The resin is filtered off, washed with DMF, dioxane, DCM and ether and then dried in the vacuum desiccator.

### Example 2

### Preparation of a Compound of the Type “V” with a Fluorine-Affine Group

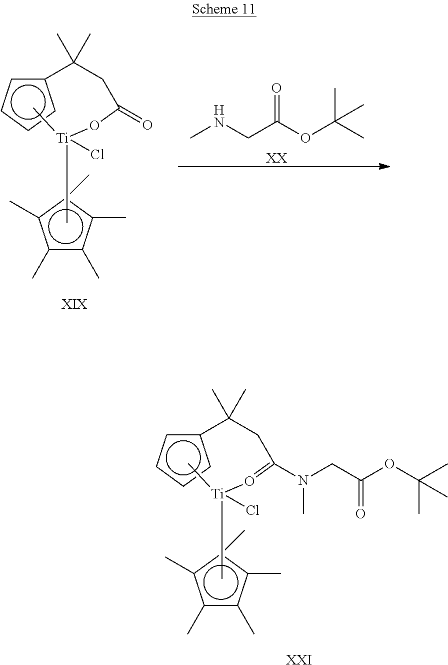

The carboxylic acid chloride of Example 2 “XVII” (Gansaeuer et al. J. Am. Chem. Soc. 2005, 127, 11622-11623) is dissolved in CH₂Cl₂ (5 mL) and transferred dropwise into a mixture of CH₂Cl₂ (5 mL), NaH (5.00 mmol) and sarcosine tert-butyl ester (1.20 mmol). The reaction mixture is stirred for 5 hours at room temperature. After filtration over celite, the filtrate is concentrated by evaporation in the vacuum. The resulting solid is taken up with CH₂Cl₂ (20 mL) and washed with a solution of 10 mL each of (1 N) NH₄Cl and NaCl. The organic phase is dried over MgSO₄ and chromatographed over silica gel 60. The product fractions are dried in the vacuum desiccator.

### Example 3

### Elimination of the Tert-Butyl Group of the Sarcosine Derivative from Example 3

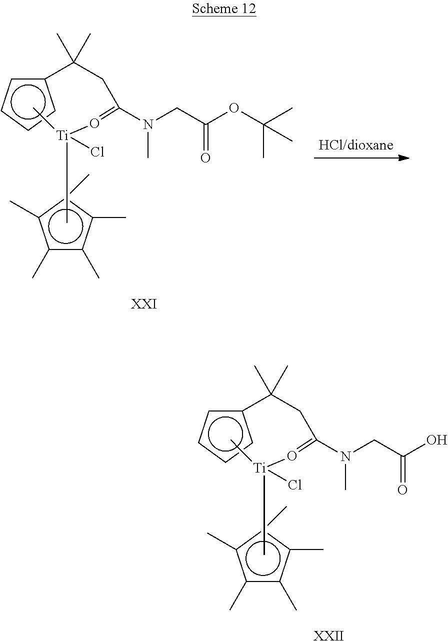

The tert-butyl ester is eliminated with a saturated solution of HCl in dioxane.

### Example 4

### Immobilization of the Compound from Example 3 on the Carrier of the Type “I” (XXIII)

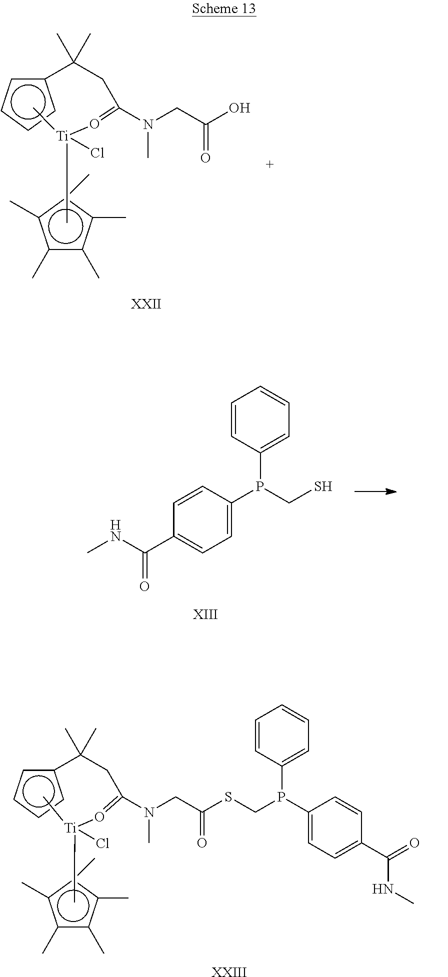

The immobilization of the compound from Example 3 takes place according to the procedure from Example 1.

### Example 5

### Fluorination of the Carrier “V” from Example 1 for the Obtainment of a Reagent with the General Formula “III” (XXV) that Contains a Fluorine-Affine Group

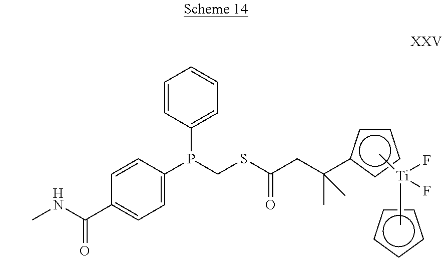

The resin from Example 2 is treated for 30 minutes with aqueous KF solution at room temperature. Then the resin is washed with water until it is salt-free.

### Example 6

### Fluorination of the Carrier “V” from Example 4 for the Obtainment of a Reagent with the General Formula “III” (XXVI) that Contains a Fluorine-Affine Group

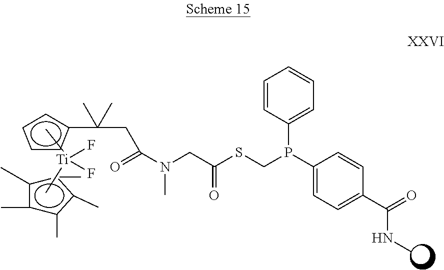

The resin from Example 5 is treated for 30 minutes with aqueous KF solution at room temperature. Then the resin is washed with water until it is salt-free.

### Example 7

### Preparation of a Compound of the Type “II” with a Fluorine-Affine Group from an Element of the 4th Group

The compound from Example 4 is treated for 30 minutes with aqueous KF solution at room temperature. Then the compound the resin is washed with water until it is salt-free.

### Example 8

### Acylation of a Compound by Means of Reagent “III” from Example 5, Wherein a Compound of the General Formula “IX” (XXVIII) is Formed

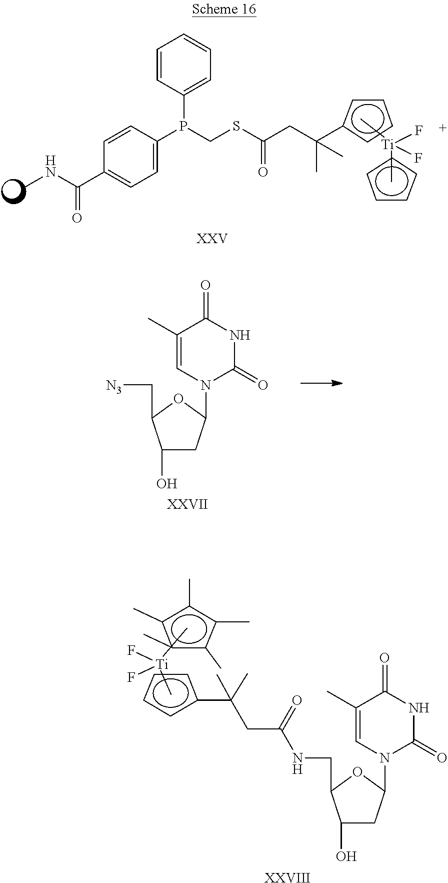

5′-Azidothymidine was reacted with reagent “III” from Example 5 by analogy with the procedure of Hanyoung Kim et al. Chem. Lett. 2006, 8, 1149-51.

### Example 9

### Acylation of an Oligonucleotide by Means of Reagent “III” from Example 6, Wherein a Compound of the General Formula “IX” (XXX) is Formed

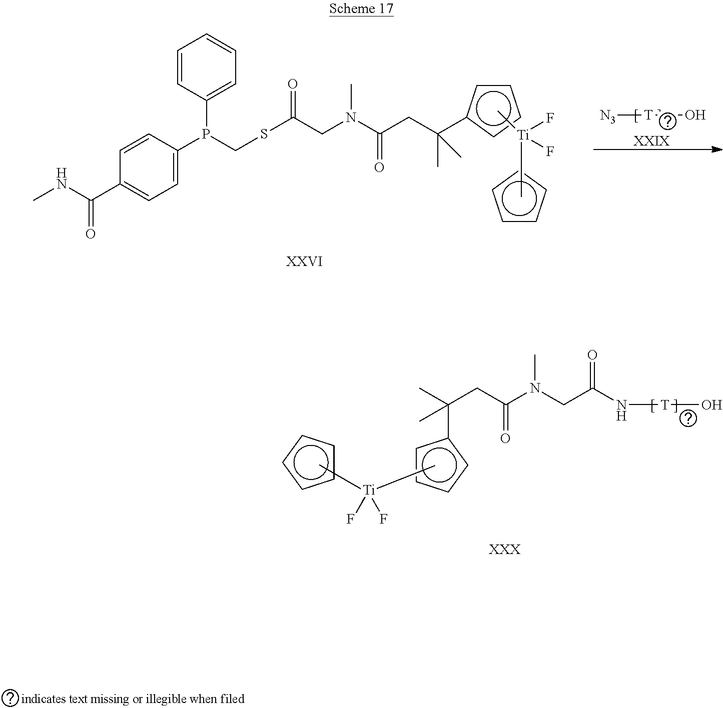

To 0.01 g of the resin from Example 6, in 2 mL of an aqueous buffer solution of 100 mg of an oligonucleotide which carries an azido group at the 5′-end is added. The fluorinated oligonucleotide diffuses out of the resin and after one hour may be isolated from the filtrate from filtration of the resin.

### Example 10

### Acylation of a Peptide by Means of Reagent “III” from Example 6, Wherein a Compound of the General Formula “IX” (XXXIII) is Formed

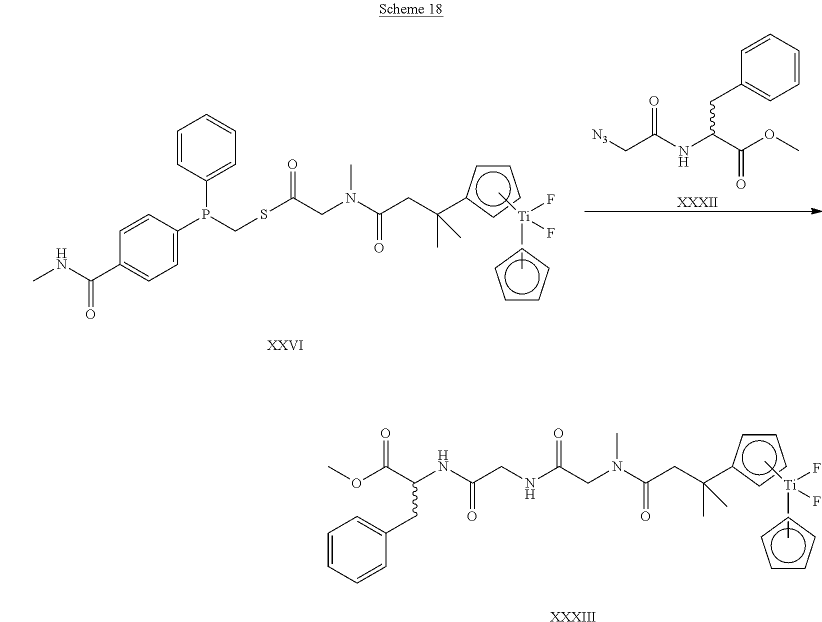

To 0.01 g of the resin from Example 6, which is contained in a column that can be closed at the outlet, in 2 mL of an aqueous buffer solution of 100 mg of a peptide which carries an azide group is added. After incubation for one hour, the outlet is opened and the fluorinated oligonucleotide is eluted with physiological NaCl solution.

1. Carrier with the general formula (V): 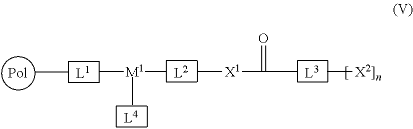
wherein
Pol stands for an insoluble organic or inorganic polymer, a metal surface or macromolecular surface;
L¹ stands for a group bound with Pol covalently or by strong ionic or Van der Waals interactions, comprising 1-200 carbon atoms and/or 0-200 heteroatoms, which are selected from the group consisting of: F, Cl, Br, I, S, O, N, Se, Te, Si, Al, Ge, P, As, Sb, B, Li, Na, K, Cs, Ca, Mg, Sr, Ba, Fe, Ce, Sn, H and the isotopes thereof;
M¹ stands for a trivalent atom that carries a free electron pair, especially P, As, Sb or Bi;
L² stands for a group comprising 1-200 carbon atoms and/or 0-200 heteroatoms, which are selected from the group consisting of: F, Cl, Br, I, S, O, N, Se, Te, Si, Al, Ge, P, As, Sb, B, Li, Na, K, Cs, Ca, Mg, Sr, Ba, Fe, Ce, Sn, H and the isotopes thereof;
L⁴ stands for a group comprising 1-200 carbon atoms and/or 0-200 heteroatoms, which are selected from the group consisting of: F, Cl, Br, I, S, O, N, Se, Te, Si, Al, Ge, P, As, Sb, B, Li, Na, K, Cs, Ca, Mg, Sr, Ba, Fe, Ce, Sn, H and the isotopes thereof;
X¹ stands for an acylatable group, especially SH, OH, NH, the silylated or stannylated derivatives thereof;
L³ stands for a fluorophilic group, which is based on an element selected from the 4th group of the periodic system consisting of titanium, zirconium, hafnium, as well as nine moieties (R¹-R⁹), which while variable independently of one another comprise H atoms or organic moieties of 1-200 carbon atoms and/or 0-200 heteroatoms, which are arrayed in aromatic rings, heteroaromatic rings, aliphatic rings, heteroaliphatic rings, aliphatic chains or heteroaliphatic chains, wherein the heteroatoms are selected from the group comprising F, Cl, Br, I, S, O, N, Se, Te, Si, Al, Ge, P, As, Sb, B, Li, Na, K, Cs, Ca, Mg, Sr, Ba, Fe, Ce;
X² stands for a group that is substitutable by fluoride, especially the following heterocycles in unsubstituted or substituted form: imidazole, triazoles, tetrazole, thiazole, benzimidazole, benzotriazole, indole, imidazoles, aniline, pyrazole, pyrrolidone, azetane, azepine, benzodiazepine, piperidine, purine, morpholine, piperazine, triazine, oxazole, hydantoin, aziridine, pyrrolidine, pyrrole, hexamethyleneimines, azaindole;
n=1-2.

2. Carrier according to claim 1, wherein X² stands for OH, SH, Cl, Br, I or a group, the conjugated organic or inorganic acids of which have a pKa<12, especially phenoxy, alkoxy, thiophenoxy or thioalkoxy groups substituted appropriately with halogens, pseudohalogens and nitro groups.

3. Carrier with the general formula (III): 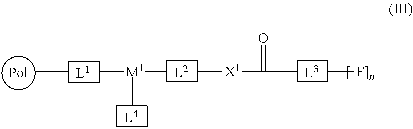
wherein
Pol stands for an insoluble organic or inorganic polymer, a metal surface or macromolecular surface;
L¹ stands for: a group bound with Pol covalently or by strong ionic or Van der Waals interactions, comprising 1-200 carbon atoms and/or 0-200 heteroatoms, which are selected from the group consisting of: F, Cl, Br, I, S, O, N, Se, Te, Si, Al, Ge, P, As, Sb, B, Li, Na, K, Cs, Ca, Mg, Sr, Ba, Fe, Ce, Sn, H and the isotopes thereof;
M¹ stands for a trivalent atom that carries a free electron pair, especially P, As, Sb or Bi;
L² stands for a group comprising 1-200 carbon atoms and/or 0-200 heteroatoms, which are selected from the group consisting of: F, Cl, Br, I, S, O, N, Se, Te, Si, Al, Ge, P, As, Sb, B, Li, Na, K, Cs, Ca, Mg, Sr, Ba, Fe, Ce, Sn, H and the isotopes thereof;
L⁴ stands for a group comprising 1-200 carbon atoms and/or 0-200 heteroatoms, which are selected from the group consisting of: F, Cl, Br, I, S, O, N, Se, Te, Si, Al, Ge, P, As, Sb, B, Li, Na, K, Cs, Ca, Mg, Sr, Ba, Fe, Ce, Sn, H and the isotopes thereof;
X¹ stands for an acylatable group, especially SH, OH, NH, the silylated or stannylated derivatives thereof;
L³ stands for a fluorophilic group, which is based on an element selected from the 4th group of the periodic system consisting of titanium, zirconium, hafnium, as well as nine moieties (R¹-R⁹), which while variable independently of one another comprise H atoms or organic moieties of 1-200 carbon atoms and/or 0-200 heteroatoms, which are arrayed in aromatic rings, heteroaromatic rings, aliphatic rings, heteroaliphatic rings, aliphatic chains or heteroaliphatic chains, wherein the heteroatoms are selected from the group comprising F, Cl, Br, I, S, O, N, Se, Te, Si, Al, Ge, P, As, Sb, B, Li, Na, K, Cs, Ca, Mg, Sr, Ba, Fe, Ce;
F stands for the ¹⁸F radioisotope;
n=1-2.

4. Bioactive substance, which binds selectively to specific cells, especially tumor cells, and contains at least one fluorinated group with the general formula (IX): 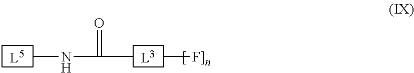
wherein
L⁵ stands for a bioactive substance, comprising an oligonucleotide consisting of 0-200 natural nucleotides or synthetic analogs and isosteres; or a synthetic polymer, which consists of 0 to 50 units; or a peptide or protein consisting of 0-2000 amino acids or the analogs, stereoisomers or isosteres thereof; or a carbohydrate consisting of 0-50 saccharide units, the analogs or stereoisomers thereof; or a low molecular weight molecule, a natural or synthetic protein agonist, antagonist, enzyme inhibitor or nucleic acid binder, or a lipid of the group of the steroids, terpenes, macrolides, polyketides and/or the natural and synthetic derivatives thereof;
L³ stands for a fluorophilic group, which is based on an element selected from the 4th group of the periodic system consisting of titanium, zirconium, hafnium, as well as nine moieties (R¹-R⁹), which while variable independently of one another comprise H atoms or organic moieties of 1-200 carbon atoms and/or 0-200 heteroatoms, which are arrayed in aromatic rings, heteroaromatic rings, aliphatic rings, heteroaliphatic rings, aliphatic chains or heteroaliphatic chains, wherein the heteroatoms are selected from the group comprising F, Cl, Br, I, S, O, N, Se, Te, Si, Al, Ge, P, As, Sb, B, Li, Na, K, Cs, Ca, Mg, Sr, Ba, Fe, Ce;
F stands for the ¹⁸F radioisotope.

5. Bioactive substance according to claim 4, wherein the fluorophilic group carries at least one fluorine atom, which is bound to a titanium atom.

6. Method for preparation of a carrier, wherein the carrier according to claim 1 is brought to reaction with ¹⁸F-labeled compounds or with ¹⁸F.

7. Method for preparation of a bioactive substance, wherein the carrier according to claim 3 is brought to reaction with an organyl azide with the general formula (VI) 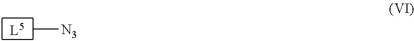
wherein
L⁵ stands for a bioactive substance, comprising an oligonucleotide consisting of 0-200 natural nucleotides or synthetic analogs and isosteres; or a synthetic polymer, which consists of 0 to 50 units; or a peptide or protein consisting of 0-2000 amino acids or the analogs, stereoisomers or isosteres thereof; or a carbohydrate consisting of 0-50 saccharide units, the analogs or stereoisomers thereof; or a low molecular weight molecule, a natural or synthetic protein agonist, antagonist, enzyme inhibitor or nucleic acid binder, or a lipid of the group of the steroids, terpenes, macrolides, polyketides and/or the natural and synthetic derivatives thereof.

8. Kit comprising a carrier according to claim 3, which is transferrable without danger while protected by its column jacketing.
